(19) United States
(12) Patent Application Publication
(10) Pub. No.: US 2025/0288318 A1
(43) Pub. Date: Sep. 18, 2025

(54) ULTRASOUND IMAGING MULTI-ARRAY SPINE IMAGING APPARATUS AND SYSTEM

(71) Applicant: Rivanna Medical, Inc., Charlottesville, VA (US)

(72) Inventors: Frank William Mauldin, Troy, VA (US); Adam Dixon, Charlottesville, VA (US); Paul Sheeran, Waxhaw, NC (US); Kathryn Ozgun, Charlottesville, VA (US)

(21) Appl. No.: 19/192,209

(22) Filed: Apr. 28, 2025

Related U.S. Application Data
(63) Continuation of application No. 18/608,412, filed on Mar. 18, 2024.
(60) Provisional application No. 63/639,221, filed on Apr. 26, 2024.

(51) Int. Cl. A61B 17/34; A61B 90/00

(52) U.S. Cl. CPC A61B 17/3403; A61B 90/37; A61B 2017/3413; A61B 2090/378

(57) ABSTRACT

An ultrasound-based scanning system and method using multiple transducer arrays and methods and non-transitory computer-readable media to optimize ultrasound visualization of spinal anatomy.

### CROSS-REFERENCE TO RELATED APPLICATIONS

The present application relies on the disclosures of and claims priority to and the benefit of the filing date of U.S. application Ser. No. 18/608,412, filed Mar. 18, 2024. The present application also relies on the disclosures of and claims priority to and the benefit of the filing date of U.S. Application No. 63/639,221, filed Apr. 26, 2024. The disclosures of the above applications are hereby incorporated by reference herein in their entireties.

### STATEMENT REGARDING FEDERALLY SPONSORED RESEARCH

This invention was made with government support under Grant No. R44NS120798 awarded by the National Institutes of Health (NIH) National Institute of Neurological Disorders and Stroke (NINDS). The government has certain rights in the invention.

### TECHNICAL FIELD

The present invention is related to an ultrasound-based scanning device and more specifically to an apparatus and method for using ultrasound-based scanning of spinal anatomy.

### BACKGROUND OF THE INVENTION

Ultrasound is increasingly used to image spinal anatomy for the purposes of diagnosis and guidance of interventional procedures including, for example, lumbar punctures, acute pain analgesia, chronic pain therapy injections, and focused ultrasound therapy. Alternative approaches for spinal imaging and therapeutic interventions include computed tomography, fluoroscopy, and magnetic resonance imaging (MRI). X-ray-based approaches, such as computed tomography or fluoroscopy, exhibit high success rates but expose the patient to ionizing radiation and increase procedure cost and are generally inaccessible at the bedside or, for interventional procedures, are incompatible with workflow constraints in fields such as emergency medicine. MRI-based approaches do not expose the patient to non-ionizing radiation, but increase procedure cost, cannot be performed in real-time, and are generally inaccessible at the bedside or, for interventional procedures, are incompatible with workflow constraints in fields such as emergency medicine. General ultrasound imaging devices and ultrasound imaging probes are designed to accommodate a wide range of applications, and the geometry of modern ultrasound imaging probes lead to sub-optimal acoustic paths for imaging spinal anatomy that obscure visualization of key anatomical features.

To overcome the limitations of current state of the art approaches to ultrasound-based spine imaging, the present invention describes a unique ultrasound-based multi-array apparatus with a physical separation between the arrays and an angled orientation relative to the spinal anatomy that provides superior feature visualization relative to conventional ultrasound imaging devices. Further, the separation between arrays within the multi-array apparatus enables incorporation of one or more therapeutic apparatus co-aligned with the imaging plane for the purpose of real-time (or substantially real-time) and/or simultaneous ultrasound image acquisition from multiple ultrasound transducer arrays during the therapeutic intervention. The invention described herein retains the benefits of medical ultrasound while addressing anatomy-specific imaging constraints that exist for spinal anatomy.

Related art describes multi-array apparatus to facilitate diagnostic and interventional procedures.

U.S. patent application Ser. No. 06/396,784, hereby incorporated by reference herein, describes an ultrasonic probe for use in needle insertion procedures, the ultrasonic probe including a support having an array of ultrasonic transducer elements lying flatwise on the front end and a groove in the support for guiding the needle. In the ultrasonic probe of U.S. patent application Ser. No. 06/396,784, the groove forms an opening at the front end of the support, and one or more transducer elements are located adjacent to the opening of the groove and between the other transducer elements, thus leaving no blank space on the front end of the support. In differentiation with the ultrasonic probe of U.S. patent application Ser. No. 06/396,784, the current invention comprises two or more angled ultrasound transducer arrays, each array having, in embodiments, an acoustic standoff that enables angling of the ultrasound transducer array. Furthermore, the current invention comprises ultrasound transducer arrays and specialized transmitting and receiving beamforming that produces overlapping two-dimensional (“2D”) images.

Japanese application JP7153980A, hereby incorporated by reference herein, describes an ultrasonic probe comprising two flat ultrasound transducer arrays having a groove between the two flat ultrasound transducer arrays, and further requires a cannula (needle) placed parallel to the primary axis of the groove. In differentiation with the ultrasonic probe of Japanese application JP7153980A, the current invention comprises two or more angled ultrasound transducer arrays, each array having, in embodiments, an acoustic standoff that enables angling of the ultrasound transducer array. Furthermore, the current invention comprises ultrasound transducer arrays and specialized transmitting and receiving beamforming that produces overlapping 2D images.

U.S. patent application Ser. No. 06/511,285, hereby incorporated by reference herein, describes an ultrasonic transducer probe comprising a flat ultrasound transducer array with a gap that can receive a removable wedge-shaped cannula (needle) adapter. In differentiation with the ultrasonic probe of U.S. patent application Ser. No. 06/511,285, the current invention comprises two or more angled ultrasound transducer arrays, each array having, in embodiments, an acoustic standoff that enables angling of the ultrasound transducer array. Furthermore, the current invention comprises ultrasound transducer arrays and specialized transmitting and receiving beamforming that produces overlapping 2D images.

PCT Application PCT/US2018/026413, hereby incorporated by reference herein, describes a system comprising an ultrasound probe, the ultrasound probe comprising two ultrasound transducers arranged at an angle that transmit sound waves to create an overlapping imaging region, and a detachable needle guide disposed between the two transducers that extends toward a target location in the overlapping imaging region. In differentiation with the ultrasonic probe of PCT Application PCT/US2018/026413, the current invention comprises two or more angled ultrasound transducer arrays and specialized transmitting and receiving beamforming to improve and/or optimize image quality in spinal applications, each array having, in embodiments, an acoustic standoff that enables angling of the ultrasound transducer array.

Application GB0307311A, hereby incorporated by reference herein, describes an ultrasound probe comprising a housing and guide for needle insertion, the guide comprising a channel located between ultrasound transducers in the housing. In differentiation with the ultrasonic probe of Application GB0307311A, the current invention comprises two or more angled ultrasound transducer arrays, each array having, in embodiments, an acoustic standoff that enables angling of the ultrasound transducer array and specialized transmitting and receiving beamforming to optimize image quality in spinal applications.

U.S. patent application Ser. No. 14/447,110, hereby incorporated by reference herein, describes an ultrasound imaging system comprising a probe with multiple apertures, each aperture configured to independently steer acoustic transmissions into a target. In the system of U.S. patent application Ser. No. 14/447,110, acoustic beams can be transmitted from different locations and at different times to simulate various insonation geometries, particularly for elastography applications. In differentiation with the system of U.S. patent application Ser. No. 14/447,110, the current invention comprises two or more physically separated and angled ultrasound transducer arrays configured to image spinal anatomy. The current invention further includes, in embodiments, a virtual apex transmit aperture technique and acoustic standoffs that allow steering of beams along non-parallel transmission paths not simply rotated about a single central axis, enabling overlapping imaging regions specifically adapted for real-time needle guidance and spinal visualization.

U U.S. patent application Ser. No. 12/748,960, hereby incorporated by reference herein, describes an ultrasonic imaging apparatus that synthesizes transmitted waves from multiple transducer elements to emulate a virtual transmission apex and enhance image resolution. In differentiation with the ultrasonic imaging apparatus of U.S. patent application Ser. No. 12/748,960, the current invention comprises two or more ultrasound transducer arrays that are physically separated and rotated within a housing, in embodiments each array including an acoustic standoff. The current invention enables overlapping imaging fields through non-coaxial steering and includes, in embodiments, adaptive beamforming based on detection of anatomical features or medical instruments in real time, which are not disclosed in or taught by U.S. patent application Ser. No. 12/748,960.

### SUMMARY OF THE INVENTION

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

In embodiments, the present invention overcomes limitations of existing ultrasound imaging systems by providing a form factor with multiple arrays, each configured to provide an optimal acoustic window of the spinal anatomical features, allowing visualization of elements of the spinal anatomy that are difficult or impossible to resolve using conventional approaches.

In embodiments, the present invention utilizes virtual apex transmit and receive beamforming strategies to optimize the detection of spinal anatomy and minimize the presence of artifacts.

In embodiments, the present invention enables real-time ultrasound acquisition and imaging guidance during interventional procedures in spinal anatomy, such as needle guidance procedures during lumbar punctures or epidurals, or other therapeutic procedures such as ablation, histotripsy, nerve stimulation, and/or hyperthermia.

In embodiments, the present invention enables multi-angle, multi-array compounding and filtering which can be used to improve the ultrasound imaging visualization of spinal anatomies, vascular anatomies, and inserted medical instruments, such as needles.

### BRIEF DESCRIPTION OF THE DRAWINGS

The accompanying drawings illustrate certain aspects of some of the embodiments of the present invention and should not be used to limit or define the invention. Together with the written description the drawings serve to explain certain principles of the invention. For a fuller understanding of the nature and advantages of the present technology, reference is made to the following detailed description of preferred embodiments and in connection with the accompanying drawings, in which:

FIG. 1 is a schematic illustration of an exemplary multi-array ultrasound probe placed over spinal anatomy that serves as the imaging target, according to an embodiment of the invention described.

FIG. 2 is a schematic illustration of an exemplary ultrasound imaging system that incorporates a multi-array ultrasound probe, according to an embodiment of the invention described herein.

FIG. 3 is a schematic illustration of an exemplary multi-array ultrasound probe with an inserted medical instrument, according to an embodiment of the invention described herein. The probe is configured such that the medical instrument is aligned with the overlapping imaging region produced by the angled transducer arrays.

FIG. 4 is a schematic illustration of a conventional ultrasound imaging configuration, depicting a central transmit beam intersecting the spinous process and resulting in reverberation artifacts generated by the probe housing.

FIG. 5 is a schematic illustration of a virtual apex transmit beam geometry, in which beams are steered to target the epidural anatomy while reducing reverberations within the probe housing, according to an embodiment of the invention described herein.

FIG. 6A, FIG. 6B, and FIG. 6C, are a schematic illustrations of the multi-array ultrasound probe positioned over spinal anatomy, illustrating (a) a virtual apex beam targeting the spinal canal and (b) Doppler data acquisition using a range gate positioned within the canal. A block diagram (c) of a basic Doppler image processing pathway is also included. These are according to embodiments of the invention described herein.

FIG. 7A and FIG. 7B are schematic and block diagrams illustration showing adaptive control of the virtual apex transmit geometry to optimize visualization of a medical instrument. The block diagram includes examples of feedback paths that adjust beamforming parameters based on (a) analytical metrics derived from in-phase and quadrature (IQ) data, (b) model fitting against stored anatomical models of generalized spinal anatomy, or (c) outputs of a machine learning model. These are according to embodiments of the invention described herein.

FIG. 8A and FIG. 8B are composite illustrations including (a) separate images acquired from the two angled arrays, (b) geometric registration of those images to a spinal anatomy model, (c) a final image produced by averaging of the separate images acquired from the two angled arrays, and (d) a final image produced by adaptively blending the separate images acquired from the two angled arrays to improve visualization of spinal anatomy. A block diagram is also shown in FIG. 8B illustrating how blending weights and registration parameters are derived and applied.

### DETAILED DESCRIPTION OF VARIOUS EMBODIMENTS OF THE INVENTION

Ultrasound imaging transducer assemblies, otherwise known as ultrasound transducer arrays, are used in a variety of medical or clinical applications to enable medical imaging functions. In this non-limiting example, an ultrasound transducer is disposed within a transducer array to deliver a pulse, tone, sequence, or programmed energy signal into a target location to be imaged. A specific example is one or more ultrasound transducer elements that deliver an ultrasound signal into a patient's body and detect a return signal so as to form an image, such as, in aspects, a computer-generated image of the target region. Different ultrasound imaging modes can be utilized, depending on a given application and design as known to those skilled in the art. The present disclosure can be used in medical ultrasound applications but is not limited to this application. Those skilled in the art will appreciate that a variety of types of transducers, signal transmitters and/or receivers, and other arrays can also benefit from the present invention, which are comprehended hereby. The preferred embodiments herein describe needle guidance. Those skilled in the art will appreciate that the present invention may be used to guide a variety of medical instruments including, but not limited to, a catheter, trocar, ablation instrument, cutting instrument, or therapy applicator. The present invention can be utilized, in a preferred embodiment, with systems and methods previously disclosed by Mauldin et al. (PCT/US2019/012622, U.S. patent application Ser. No. 17/950,399, U.S. patent application Ser. No. 18/608,412, U.S. patent application Ser. No. 18/913,791, and U.S. patent application Ser. No. 18/608,260), which are incorporated by reference herein.

In an embodiment of the present invention for medical applications of spine imaging described herein, a probe containing two or more ultrasound transducer arrays, otherwise termed a multi-array probe, is described. In embodiments, an objective of the multi-array ultrasound probe and transmit and/or receive beamforming configuration is to provide an optimal or improved acoustic configuration to visualize key anatomical structures by arranging the array elements within a probe housing and timing energy transmitted to and received from the elements so that the acoustic energy transmitted from the arrays into the body and the acoustic reflections received from structures within the spinal anatomy are minimally obscured by surrounding anatomical structures. In embodiments, additional objectives include but are not limited to algorithmic contrast enhancement of anatomical structures and/or medical instruments (e.g., needles) within the ultrasound image. In embodiments, images acquired from each of the ultrasound transducer arrays in the multi-array probe may be compounded, such as by averaging, weighted averaging, or based on measurements of similarity, or based on detection of key features within each image in order to form composite images with a wider field of view and containing an overlapping image region that depicts the spinal anatomy.

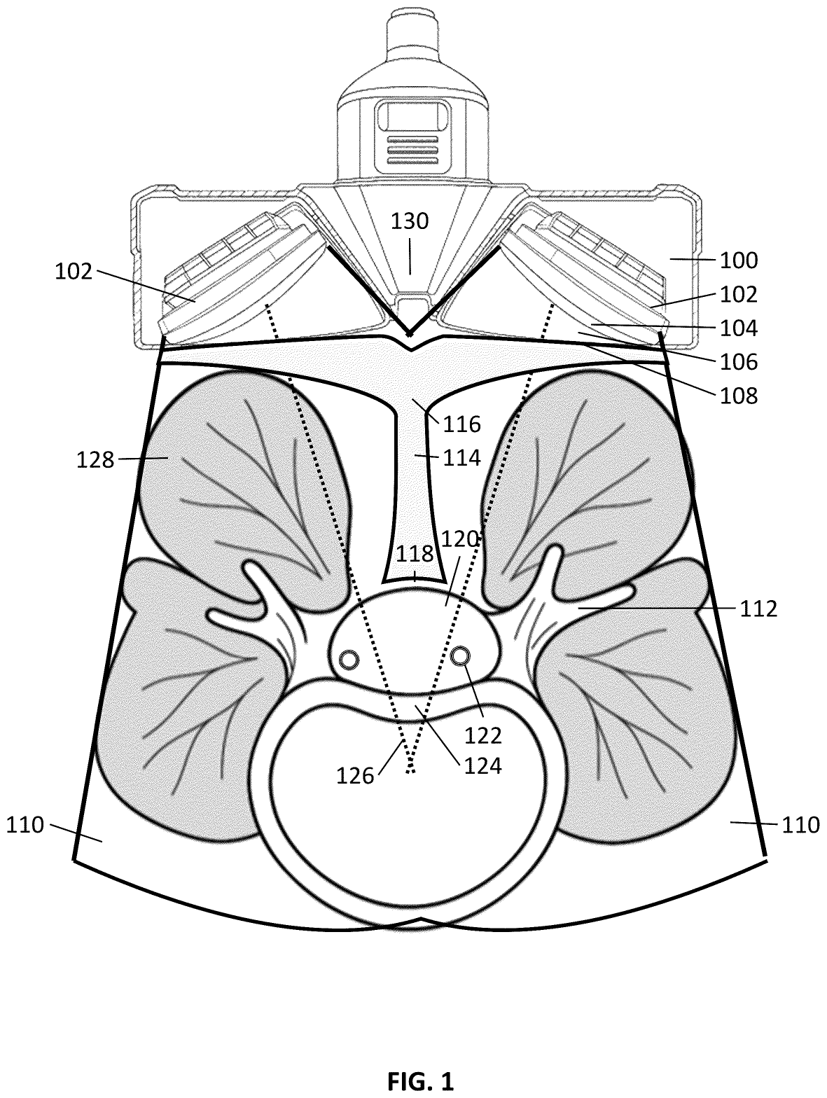

In one exemplary embodiment, the multi-array ultrasound probe is depicted in FIG. 1. In this embodiment, the multi-array probe has a probe housing 100 containing at least two ultrasound arrays 102 that interface to ultrasound data acquisition electronics via integrated circuits and an electrical signal cable. The multi-array ultrasound probe is configured such that the two ultrasound arrays 102 do not make direct contact with the patient anatomy, although contacting the patient anatomy is envisioned. The two ultrasound transducer arrays 102 acoustically couple to the patient anatomy through, in aspects, three optionally intervening acoustically transmissive layers 104, 106, and 108, although more or fewer intervening acoustically transmissive layers are envisioned. In this embodiment, the outermost acoustically transmissive layer 108 makes contact with the patient anatomy or a probe sheath, and is comprised of a rigid, semi-rigid, or substantially rigid material, such as a plastic or elastomer. In this embodiment, the central acoustically transmissive layer 106 provides an acoustic ‘filler’ between the outermost transmissive layer 108 and the innermost transmissive layer 104, and may be comprised of a rigid, semi-rigid, or substantially rigid material, such as a plastic or elastomer, a deformable, semi-deformable, or substantially deformable material, such as an elastomer or a gel, or a fluid. In this embodiment, the innermost acoustically transmissive 104 layer provides a coating to protect the ultrasound transducer array, and may be comprised of a rigid, semi-rigid, or substantially rigid material, such as a plastic or elastomer, a deformable, semi-deformable, or substantially deformable material, such as an elastomer. The ultrasound arrays 102 can be rotated and separated within the probe housing 100 in order to provide acoustic imaging sectors 110 that sample spinal anatomy. Highly attenuating elements of the spinal anatomy relevant to this invention include, by way of example, bony projections of the vertebral body 112, the interspinous ligament 114, and supraspinous ligament 116. The ultrasound arrays 102 of the multi-array probe can be configured spatially so as to provide overlapping acoustic windows 110 that sample elements of the spinal anatomy relevant to diagnosis and procedure guidance, including the posterior complex 118, the spinal canal 120, the spinal arteries 122, and the anterior complex 124, in an example. The rotation and separation of the ultrasound arrays 102 can provide a primary acoustic path 126 that avoids the highly attenuating spinal ligaments 114 and 116 while passing through the less attenuating muscle 128 that surrounds the spinal anatomy. In comparison to conventional ultrasound probes that are positioned directly above the spinal anatomy, the configuration of the present invention can provide unexpectedly superior visualization of the posterior spinal complex 118, anterior complex 124, and flow motion from intraspinal arteries 122. In some embodiments, the ultrasound arrays 102 are separated (e.g., distance-separated) by a gap, such as a gap or space of about 1 mm 130, in the housing component 100 in order to allow one or more medical instruments access to the patient anatomy for the purpose of image guidance.

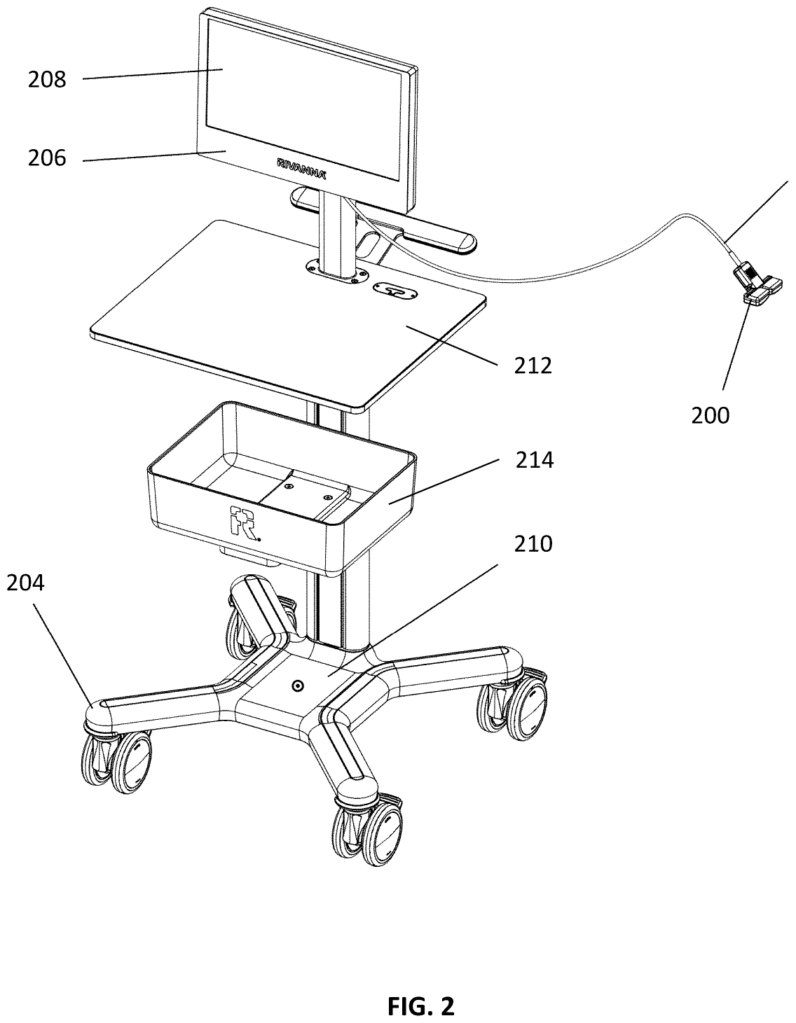

In one exemplary embodiment depicted in FIG. 2, the multi-array ultrasound probe 200 can be connected by an electrical signal cable 202 (or wirelessly) to a mobile cart 204 to allow the imaging device to be moved to the bedside and positioned at an orientation for acquiring images of the patient's anatomy. The cart 204 may contain a computer processor and/or a controller (or the processor or the controller shown can be located remotely) and monitor 206, battery, and other associated electronics familiar to those skilled in the art which are needed to power and communicate with the imaging device 200. The cart 204 can be outfitted with additional input/output devices such as a keyboard, mouse, or monitor 206, which may also be a touchscreen display, such as an electronic display 208. The cart can be outfitted with a worksurface 212 and compartment 214 in order to place or store materials commonly used during interventional procedures, such as consumables, needles, ultrasound gel, and disinfectant wipes. The monitor 206 may be positionally adjustable about the cart in order to orient the imaging device 200 and monitor 206 in various relative positions, heights, and orientations for the needle guidance procedure. In a preferred embodiment, the monitor/monitor enclosure 206 may simultaneously contain electronic display screen 208, the computer processor, the controller, and the ultrasound front-end electronics. A computer processor within the enclosure 206 may be used to perform ultrasound signal and image processing steps required to form an ultrasound image reconstruction that can be displayed on the monitor 208, although the processor may be located remotely, such an on another computer device, a server, or the cloud. Such processing steps may include but are not limited to: beamforming, bandpass filtering, scan conversion, spatial compounding, Doppler imaging, and image rendering.

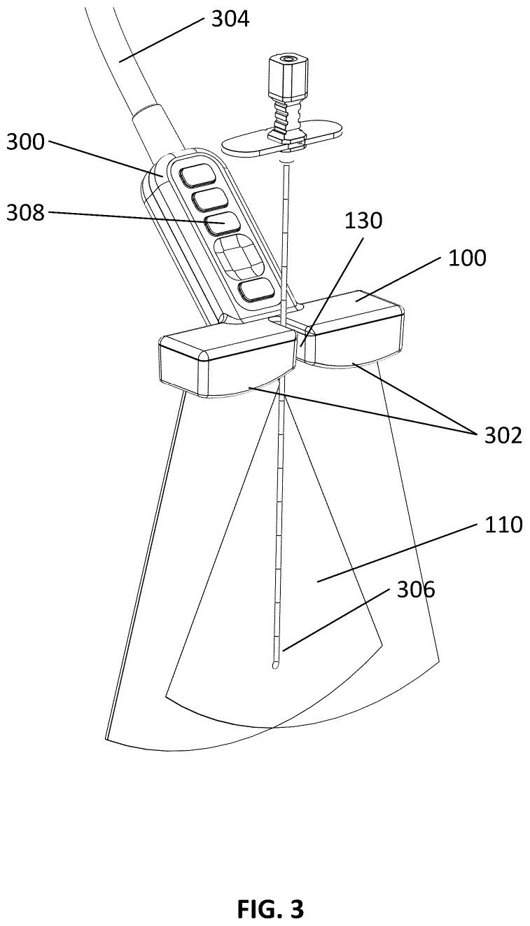

In another exemplary embodiment, the multi-array ultrasound probe 100 is depicted in FIG. 3. The multi-array probe 100 has a housing component that provides a handle 300 with grip features for holding the probe. In embodiments, the multi-array probe 100 can incorporate two ultrasound arrays 302 that interface to ultrasound data acquisition electronics via integrated circuits and an electrical signal cable 304 to acquire ultrasound image datasets. The ultrasound arrays 302 of the multi-array probe can be separated by a medical instrument guide 130 comprising a gap or space integral to the probe housing that provides access for a central needle trajectory 306 that passes between the two ultrasound arrays 302. In this imaging configuration, the fields of view (“FOV”) 110 of the two ultrasound arrays 302 overlap along the needle trajectory 110, providing for independent views of the needle from two different vantage points. In some embodiments, the medical instrument guide 130 provides alignment to keep the central needle trajectory 306 in plane with the fields of view (FOV) 110 of the two ultrasound arrays while also minimizing forces imparted to the instrument, which may be advantageous for clinical procedures where the clinician relies on the tactile feedback of the instrument passing through the anatomy, such as for detecting loss of resistance. In some embodiments, the medical instrument guide 130 allows a needle angulation of up to and including 20 degrees relative to the central axis of the medical instrument guide 130, although smaller and larger angulations are envisioned. The dual-array probe 100 can incorporate buttons 308 (or other physical or digital interfaces) to provide control over imaging functionality. The handle 300 can include a printed circuit board which provides a microprocessor and/or a controller that interfaces to buttons 308, or other physical controls, to provide a motion sensor integrated circuit, and enable digital serial communication through the probe cable 304 to the host processor of the ultrasound imaging system.

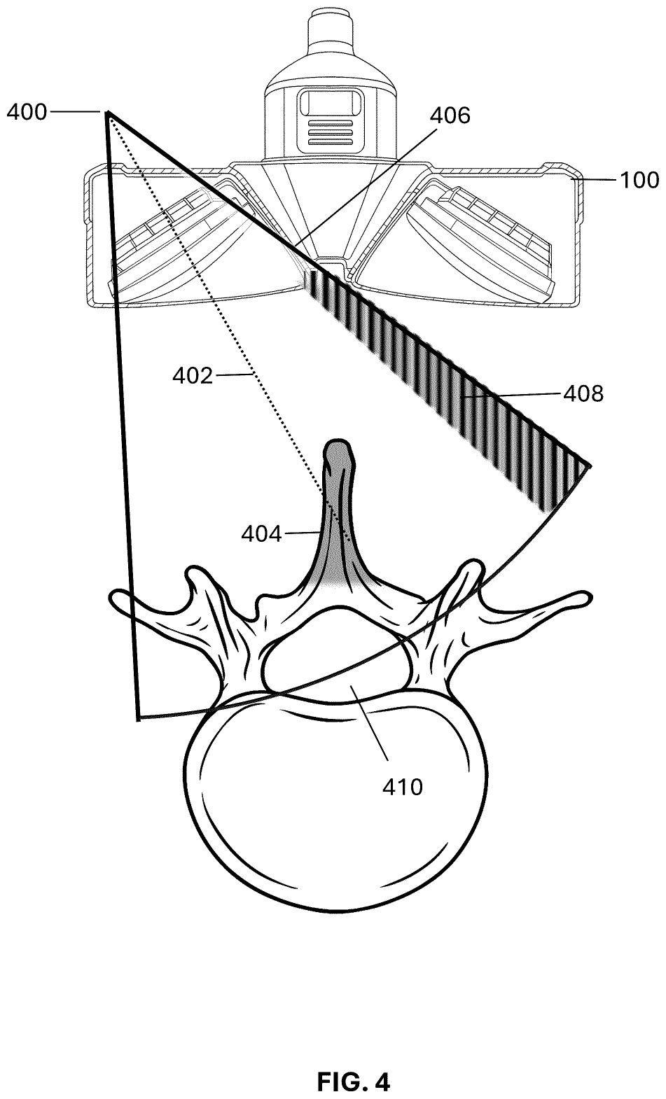

FIG. 4 illustrates a currently-existing dual-array probe with a conventional convex array transmit and receive beamforming configuration, in which a central ultrasound beam 402 originates from a conventional geometric center 400. In this imaging configuration the transmitted acoustic energy is directed along a fixed path that may intersect with anatomical structures such as the spinous process 404, depending on the specific anatomical presentation of the subject being imaged (e.g., the depth of the spine within the tissue). As a result of rotating the arrays within the probe housing 100, a significant amount of acoustic energy is directed into the probe housing interior 406 and is reflected, causing internal reverberation and image degradation. This known configuration also manifests as a region of image dropout 408 posterior to the probe, as energy cannot be transmitted through the probe housing to reach this imaging region. The acoustic interference and central ultrasound beam path observed in this embodiment limits the ability to resolve the spinal canal 410 and is similar to limitations inherent in conventional scanning geometries positioned directly over the midline of the spine. As such, both conventional embodiments of probe architectures as well as some embodiments of dual-array probe architectures and transmit/receive beamforming provide non-ideal views of the anatomical structures pertinent for procedure guidance; accordingly, the invention presented herein is an improvement over known devices and techniques described in this paragraph and as shown in FIG. 4.

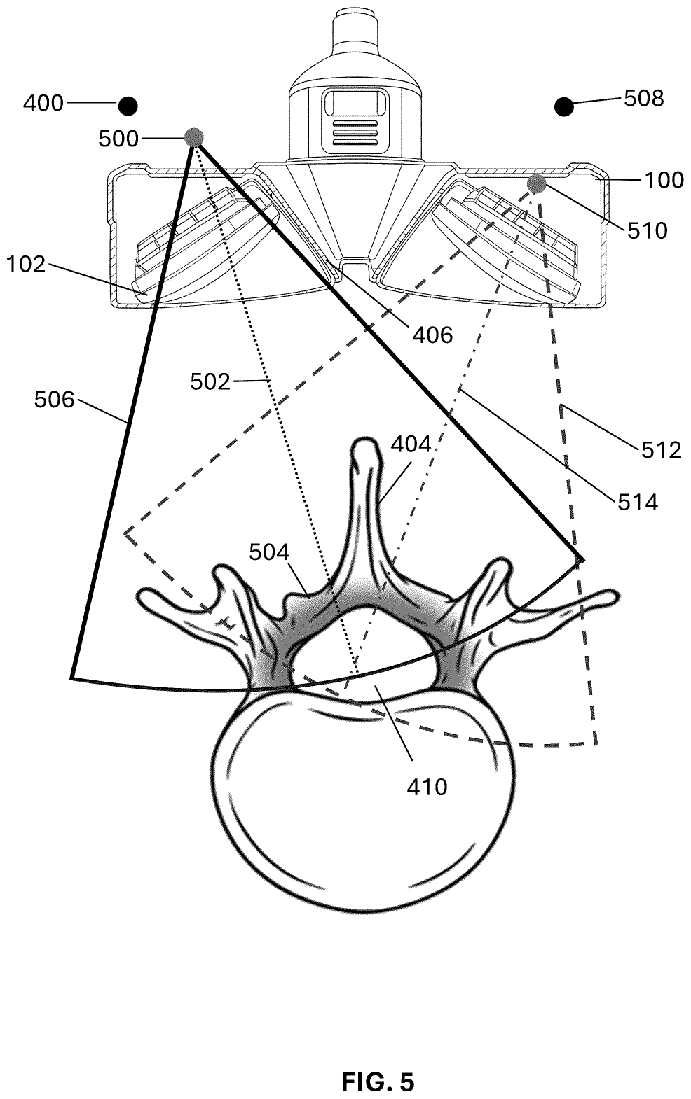

FIG. 5 presents an embodiment according to the current invention of a dual-array probe employing a virtual apex beamforming geometry in order to mitigate the drawbacks of conventional probe geometries and dual-array probe geometries already existing in the art, such as shown in FIG. 4. In the embodiment in FIG. 5, the ultrasound system generates beams as if they emanate from a virtual origin point, also referred to herein as a virtual apex point or virtual apex point location 500, rather than the geometric probe origin 400, which is defined by the geometry of each ultrasound transducer array in the dual-array probe. The central acoustic beam 502 in this embodiment is directed to intersect spinal anatomy (e.g., target epidural anatomy) 504 in order to capture optimal or improved views of the spinal canal 410 while avoiding acoustic interaction with the probe housing 100 or housing for the separate ultrasound transducer arrays 406 and minimizing attenuation by the spinous process 404. The sector span 506 that is imaged by the ultrasound transducer array is defined by the virtual apex point location 500 relative to the ultrasound transducer array 102. In embodiments, the second ultrasound transducer array may have a differing virtual apex point location 510 relative to the second ultrasound transducer array's geometric apex 508, which can be the same or different than, for example, the configuration and/or virtual apex transmit beam geometry of the first ultrasound transducer array. The central acoustic beam of the second ultrasound transducer array 512 in this embodiment is directed to intersect spinal anatomy, and the sector span 514 that is imaged by the second ultrasound transducer array is defined by the virtual apex point location 510 relative to the second ultrasound transducer array. By way of example, FIG. 5 depicts a virtual apex point location 500 for the first array that is just behind the probe housing 100 but differs from the ultrasound transducer array's geometric apex 400, while a virtual apex point location 510 for the second array is just inside the probe housing 100 and differs from the second array's geometric apex 508. By reconfiguring the transmit origin and steering direction, this embodiment enables access to clinically relevant regions of the spine while substantially reducing reverberation artifacts caused by probe housing structures. In embodiments, the location of the virtual origin point 500 can be configured by the ultrasound system beamformer and/or processor to provide optimal or improved/enhanced views of particular features of the spinal anatomy. In embodiments, a processor and/or beamformer may adaptively alter the location of the virtual origin point 500 on the basis of a medical instrument detection algorithm for the purpose of enhancing visualization of an inserted medical instrument. In embodiments, a processor and/or beamformer may adaptively alter the location of the virtual origin point 500 on the basis of image quality metrics derived from the ultrasound data. In embodiments, the image quality metrics can be used as inputs to adaptively alter the location of the virtual origin point 500, and, in aspects, may be derived from comparison(s) between acquired ultrasound images and/or data and a pre-acquired anatomical model, such as a pre-acquired patient anatomical model. In embodiments, the image quality metrics used as inputs to adaptively alter the location of the virtual origin point 500 may be derived from the output of an artificial intelligence or machine learning model that receives as input the acquired ultrasound data. In aspects, the invention, as explained herein, (a) can generate and/or change a location of the virtual apex point location 500, (b) it can select and/or change/adjust one or more acoustic transmission powers, (c) it can select and/or adjust/change one or more acoustic transmission angles (e.g., 502), such as acoustic beam angles (e.g., 502). In aspects, while only depicted on the left side in FIG. 5, the same generation, control, changes, and adjustments can occur in the other ultrasound transducer array of the ultrasound probe, such as the array depicted on the right of the probe 100 in FIG. 5. In other embodiments, there can be even more than two ultrasound transducer arrays, and these inventive elements can apply to those ultrasound transducer arrays, as well.

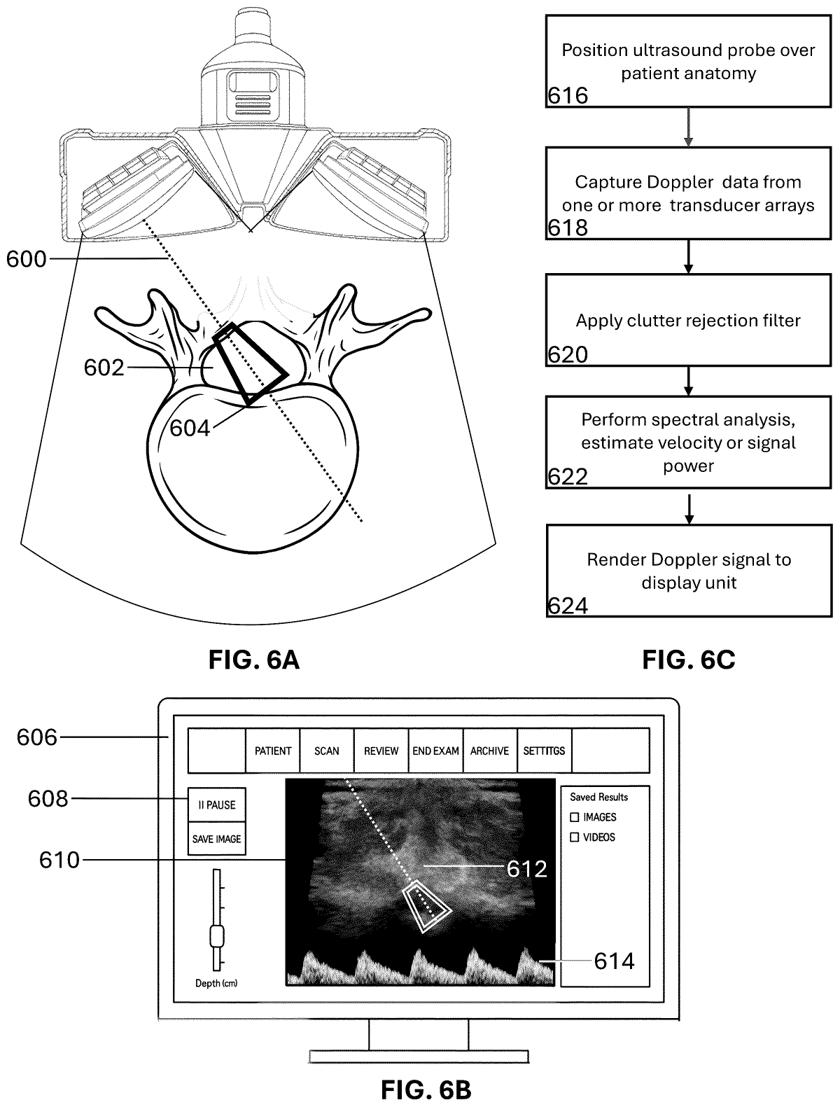

An embodiment utilizing the virtual apex configuration for Doppler data acquisition is illustrated in FIGS. 6A, 6B, and 6C. In FIG. 6A, a central acoustic beam 600 and its angle are steered through the spinal canal 602, and Doppler signals are acquired from a range gate 604 positioned within vascular anatomy of interest. In embodiments depicted in FIG. 6B, the system display 606 presents a scan page 608 that includes a real-time or substantially real-time B-mode image 610 and a visual range gate icon 612, which the user may manipulate to align with target spinal anatomy, anatomical structures, and/or flow structures. In embodiments, a spectral Doppler signal 614 is rendered in real-time (or substantially real-time) based on motion detected within the selected range. In an embodiment, FIG. 6C depicts an acquisition workflow that involves positioning the ultrasound probe over patient anatomy 616, capturing Doppler data from one or more transducer arrays 618, applying a clutter rejection filter 620, performing spectral analysis to estimate velocity or signal power 622, and rendering the Doppler signal to the display unit 624. In embodiments, the system may evaluate acoustic sensitivity and reconfigure the transmit geometry to improve visibility of the feature in response to suboptimal visualization, producing optimized image formation and flow estimations.

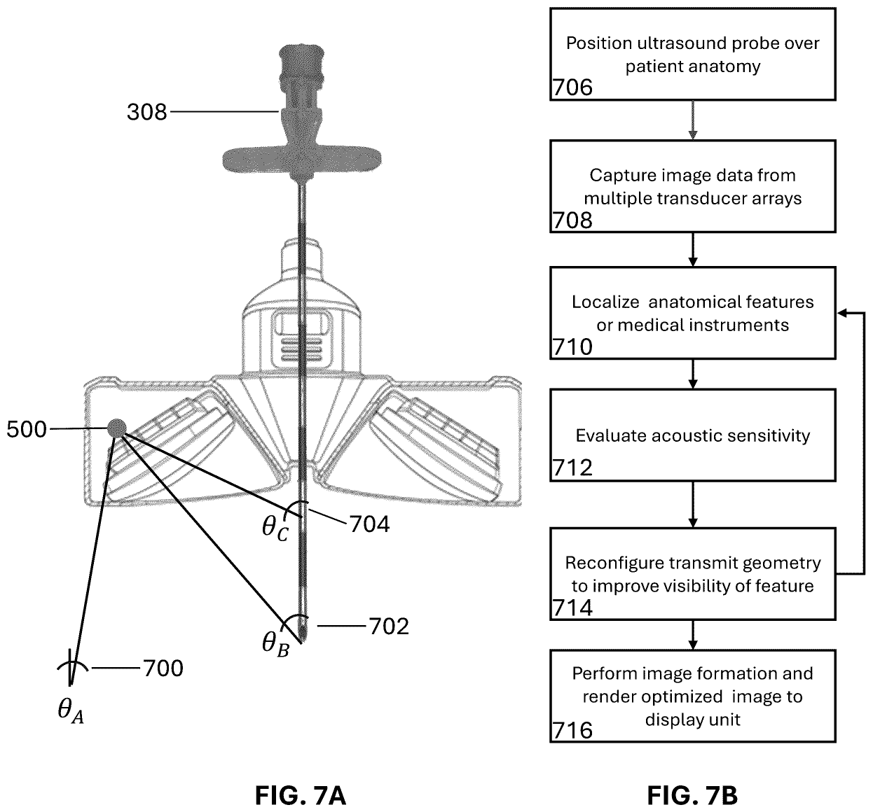

FIGS. 7A and 7B depict an embodiment in which the virtual apex (beamforming) geometry/angle/location or virtual apex point location is adaptively controlled to enhance visualization of a medical instrument 308. In embodiments, the virtual apex origin point 500 is dynamically adjusted/moved to provide a scan field of view 700 with a desired angle of incidence relative to an interventional tool while simultaneously minimizing energy deposition along acoustic paths that may produce reverberant echoes. In FIG. 7A, angle θ_{A} 700 represents an outer angle defining the desired outer extent of the imaging sector, OB 702 represents an angle of incidence relative to the tip of the medical instrument at a given insertion depth, and θ_{C} 704 represents an inner angle defining the beamforming limit beyond which reverberation from the probe housing will occur. In embodiments, the virtual apex beamforming configuration is adjusted by constraining θ_{A} and θ_{C} and adjusting parameters to optimize or improve/enhance OB. The beamforming parameters in this embodiment may be adjusted in real-time or substantially real-time based on one or more feedback pathways described above. In embodiments, these include but are not limited to analytical metrics derived from in-phase and quadrature (IQ) data, model fitting against stored anatomical models of generalized spinal anatomy, and outputs from a machine learning model trained to optimize or enhance/improve visualization of anatomical features and procedural instruments. In embodiments, the adjustment of beamforming parameters provides enhanced image guidance during procedures requiring precise instrument placement, including epidural access and other spinal interventions. In embodiments, such as that shown in FIG. 7A, the two (or more) ultrasound transducer arrays (elements 102 in FIG. 1) are oriented at selected rotation angles within the probe housing (element 100 in FIG. 1) such that a central acoustic axis or axes of the two or more ultrasound transducer arrays shown in FIG. 7A, by way of example, intersect to create an overlapping acoustic imaging region configured for imaging the spinal anatomy. In aspects, the two or more ultrasound transducer arrays are rotated within the probe housing so that the central axis of each of the two or more ultrasound transducer arrays are angled relative to a contact surface of the probe housing that couples with or is placed on or near patient anatomy. The processor shown in the figures (or located remotely) can be configured to operatively generate one or more virtual apex point locations (see, e.g., element 500) and control acoustic transmissions, including one or more acoustic transmission powers, one or more acoustic transmission angles, or both, from the two or more ultrasound transducer arrays using virtual apex point location generation and acoustic transmission adjustment. In aspects, the processor may be capable of operatively (a) generating the one or more virtual apex point locations (e.g., 500) (b) selecting one or more acoustic transmission powers, (c) selecting one or more acoustic transmission angles shown in the figures (e.g., element 502 in FIG. 5), or (d) combinations thereof, based on a patient's anatomy or one or more anatomical models of generalized spinal anatomy, to remove or decrease internal acoustic reverberations or image degradation (see, e.g., element 408 in FIG. 4). In aspects, the acoustic transmission adjustment comprises steering acoustic beams (e.g., elements 502 and 514 in FIG. 5) from the two pictured ultrasound transducer arrays along one or more transmission axes starting at the one or more generated virtual apex point locations in addition to or beyond rotations of each ultrasound transducer array about each ultrasound transducer array's central axis, thereby creating real-time or substantially real-time visualization of spinal anatomical structures/spinal anatomy, enhancement of visualization of one or more medical instruments inserted within or near the spinal anatomy, and minimizing internal acoustic reverberations, image degradation, or both. Optionally, the one or more virtual apex points change location, the one or more acoustic transmission powers are adjusted, the one or more acoustic transmission angles (acoustic beam angles) are adjusted, or combination thereof, based on ultrasound data received by the processor.

FIG. 7B depicts a workflow to optimize or enhance/improve image formation involving positioning the ultrasound probe over a patient anatomy, such as placing the probe housing on or near a patient contact surface, such as a patient's lower back 706, capturing image data from multiple transducer arrays 708, localizing anatomical features or visibility of medical instruments 710, evaluating acoustic sensitivity 712, reconfiguring/adjusting transmit geometry to improve visibility of a feature/spinal anatomy/anatomical structure 714, and performing image formation and rendering the optimized or enhanced/improved image to the display unit 716.

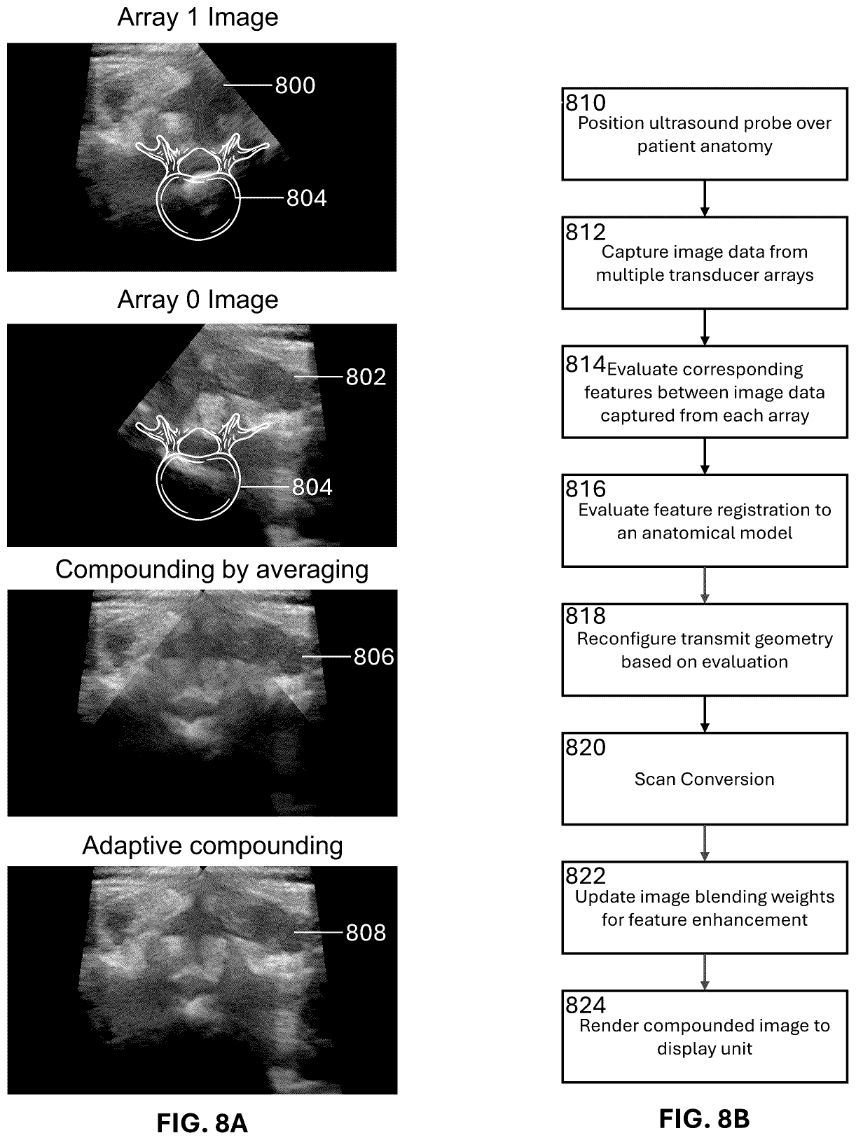

An embodiment directed toward improved image compounding is shown in FIG. 8. In this embodiment, separate images 800, 802 acquired from, by way of example, a dual-array probe, are geometrically registered to a spinal anatomy model 804 to assess feature correspondence. A compounded image 806, which is produced by taking the average of the two fields of view (or in cases, more than two fields of view), results in the appearance of interference artifacts due to misalignment between anatomical landmarks across the imaging planes. In embodiments, the system applies an adaptive blending process that derives blending weights and registration parameters based on image-model alignment in order to produce an image with improved image quality 808 and accuracy in depicting anatomical features. In embodiments, the adaptive blending may be performed on the bases of image quality metrics resulting from blending of the acquired images. In embodiments, the adaptive blending may be performed by an artificial intelligence or machine learning model. In embodiments, the adaptive blending may incorporate adjustment to the estimated speed of sound of the tissue to improve the spatial registration of features. FIG. 8B depicts a processing workflow involving positioning the ultrasound probe over patient anatomy 810, capturing/acquiring image data from two or more ultrasound transducer arrays 812, evaluating corresponding features between image data captured from each array 814, assessing registration against an anatomical model of spinal anatomy 816, reconfiguring the transmit geometry 818, applying scan conversion 820, updating the image blending weights for feature enhancement 822, and rendering the resulting image to the display unit 824.

Embodiments of the invention also include a computer readable medium comprising one or more computer files comprising a set of computer-executable instructions for performing one or more of the calculations, steps, processes, and operations described and/or depicted herein. In exemplary embodiments, the files may be stored contiguously or non-contiguously on the computer-readable medium. Embodiments may include a computer program product comprising the computer files, either in the form of the computer-readable medium comprising the computer files and, optionally, made available to a consumer through packaging, or alternatively made available to a consumer through electronic distribution. As used in the context of this specification, a “computer-readable medium” is a non-transitory computer-readable medium and includes any kind of computer memory such as floppy disks, conventional hard disks, CD-ROM, Flash ROM, non-volatile ROM, electrically erasable programmable read-only memory (EEPROM), and RAM. In exemplary embodiments, the computer readable medium has a set of instructions stored thereon which, when executed by a processor, cause the processor to perform tasks, based on data stored in the electronic database or memory described herein. The processor may implement this process through any of the procedures discussed in this disclosure or through any equivalent procedure.

In other embodiments of the invention, files comprising the set of computer-executable instructions may be stored in computer-readable memory on a single computer or distributed across multiple computers. A skilled artisan will further appreciate, in light of this disclosure, how the invention can be implemented, in addition to software, using hardware or firmware. As such, as used herein, the operations of the invention can be implemented in a system comprising a combination of software, hardware, or firmware.

Embodiments of this disclosure include one or more computers or devices loaded with a set of the computer-executable instructions described herein. The computers or devices may be a general purpose computer, a special-purpose computer, or other programmable data processing apparatus to produce a particular machine, such that the one or more computers or devices are instructed and configured to carry out the calculations, processes, steps, operations, algorithms, statistical methods, formulas, or computational routines of this disclosure. The computer or device performing the specified calculations, processes, steps, operations, algorithms, statistical methods, formulas, or computational routines of this disclosure may comprise at least one processing element such as a central processing unit (i.e., processor) and a form of computer-readable memory which may include random-access memory (RAM) or read-only memory (ROM). The computer-executable instructions can be embedded in computer hardware or stored in the computer-readable memory such that the computer or device may be directed to perform one or more of the calculations, steps, processes and operations depicted and/or described herein.

Additional embodiments of this disclosure comprise a computer system for carrying out the computer-implemented method of this disclosure. The computer system may comprise a processor for executing the computer-executable instructions, one or more electronic databases containing the data or information described herein, an input/output interface or user interface, and a set of instructions (e.g., software) for carrying out the method. The computer system can include a stand-alone computer, such as a desktop computer, a portable computer, such as a tablet, laptop, PDA, or smartphone, or a set of computers connected through a network including a client-server configuration and one or more database servers. The network may use any suitable network protocol, including IP, UDP, or ICMP, and may be any suitable wired or wireless network including any local area network, wide area network, Internet network, telecommunications network, Wi-Fi enabled network, or Bluetooth enabled network. In one embodiment, the computer system comprises a central computer connected to the internet that has the computer-executable instructions stored in memory that is operably connected to an internal electronic database. The central computer may perform the computer-implemented method based on input and commands received from remote computers through the internet. The central computer may effectively serve as a server and the remote computers may serve as client computers such that the server-client relationship is established, and the client computers issue queries or receive output from the server over a network.

The input/output interfaces may include a graphical user interface (GUI) which may be used in conjunction with the computer-executable code and electronic databases. The graphical user interface may allow a user to perform these tasks through the use of text fields, check boxes, pull-downs, command buttons, and the like. A skilled artisan will appreciate how such graphical features may be implemented for performing the tasks of this disclosure. The user interface may optionally be accessible through a computer connected to the internet. In one embodiment, the user interface is accessible by typing in an internet address through an industry standard web browser and logging into a web page. The user interface may then be operated through a remote computer (client computer) accessing the web page and transmitting queries or receiving output from a server through a network connection.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Other embodiments of the invention will be apparent to those skilled in the art from consideration of the specification and practice of the invention.

It is noted that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

As used herein, the term “about” refers to plus or minus 5 units (e.g., percentage) of the stated value.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, the term “substantial” and “substantially” refers to what is easily recognizable to one of ordinary skill in the art.

It is to be understood that the phraseology and terminology employed herein is not to be construed as limiting and are for descriptive purpose only.

It is to be understood that while certain of the illustrations and figure may be close to the right scale, most of the illustrations and figures are not intended to be of the correct scale.

It is to be understood that the details set forth herein do not construe a limitation to an application of the invention.

Furthermore, it is to be understood that the invention can be carried out or practiced in various ways and that the invention can be implemented in embodiments other than the ones outlined in the description above.

As used herein, the term “medical instruments” refers to needles, catheters, trocars, ablation instruments, cutting instruments, and therapy applicators.

1) An ultrasound imaging system for imaging a spinal anatomy comprising:
a probe housing comprising two or more ultrasound transducer arrays;
wherein each ultrasound transducer array of the two or more ultrasound transducer arrays is oriented at selected rotation angles within the probe housing such that a central acoustic axis or axes of the two or more ultrasound transducer arrays intersect to create an overlapping acoustic imaging region configured for imaging the spinal anatomy;
wherein the two or more ultrasound transducer arrays are rotated within the probe housing so that the central axis of each of the two or more ultrasound transducer arrays are angled relative to a contact surface of the probe housing that couples with or is placed on or near a patient contact surface; and
a processor configured to operatively generate one or more virtual apex point locations and control acoustic transmissions from the two or more ultrasound transducer arrays using virtual apex point location generation and acoustic transmission adjustment;
wherein the processor operatively generates the one or more virtual apex point locations based on the spinal anatomy or one or more anatomical models of a generalized spinal anatomy, to remove or decrease internal acoustic reverberations, image degradation, or both; and
wherein the acoustic transmission adjustment comprises transmitting acoustic beams from two or more ultrasound transducer arrays of the two or more ultrasound transducer arrays along one or more transmission axes starting at the one or more generated virtual apex point locations, thereby creating real-time or substantially real-time visualization of the spinal anatomy, enhancement of visualization of one or more medical instruments inserted within or near the spinal anatomy, and minimizing internal acoustic reverberations, image degradation, or both.

2) The system of claim 1, wherein the one or more virtual apex point locations do not coincide with, or wherein the one or more virtual apex locations are different than, any of the physical apexes of the one or more ultrasound transducer arrays, as defined by their geometry.

3) The system of claim 1, wherein the one or more virtual apex point locations change a location based on ultrasound data received by the processor.

4) The system of claim 1, wherein the controlled acoustic transmissions include controlling one or more acoustic transmission powers, and wherein the configured processor operatively selects the one or more acoustic transmission powers based on the spinal anatomy or the one or more anatomical models of generalized spinal anatomy, to remove or decrease the internal acoustic reverberations, the image degradation, or both, wherein the one or more transmission powers are automatically adjusted based on ultrasound data received by the processor.

5) The system of claim 1, wherein the controlled acoustic transmissions include controlling one or more acoustic transmission angles, and wherein the configured processor operatively selects the one or more acoustic transmission angles based on the spinal anatomy or the one or more anatomical models of generalized spinal anatomy, to remove or decrease the internal acoustic reverberations, the image degradation, or both, and wherein the one or more acoustic transmission angles are automatically adjusted based on ultrasound data received by the processor.

6) The system of claim 1, wherein each of the two or more ultrasound transducer arrays is physically separated by at least about 1 mm.

7) The system of claim 1, further comprising at least one acoustically transmissive standoff layer positioned between each of the two or more ultrasound transducer arrays and the patient contact surface.

8) The system of claim 7, wherein the at least one acoustically transmissive standoff layer has acoustic impedance characteristics matched or substantially matched within +/−50% of soft tissue, and wherein the at least one acoustically transmissive standoff layer provides an angled patient interface to minimize internal acoustic reverberations.

9) The system of claim 7, wherein the at least one acoustically transmissive standoff layer comprises a patient contact interface and an acoustic filler material.

10) The system of claim 1, wherein a first central axis of acoustic propagation from a first ultrasound transducer array of the two or more ultrasound transducer arrays and a second central axis of acoustic propagation from a second ultrasound transducer array of the two or more ultrasound transducer arrays are not aligned or co-aligned with an interspinous ligament or a spinous process when the probe housing is centered over all or part of the spinal anatomy, such as in a transverse view.

11) The system of claim 1, wherein a first central axis of sound propagation from a first ultrasound transducer array of the two or more ultrasound transducer arrays differs from a second central axis of sound propagation from a second ultrasound transducer array of the two or more ultrasound transducer arrays.

12) The system of claim 1, wherein the configured processor is further operative to receive ultrasound data from the two or more ultrasound transducer arrays or from beamforming electronics that receive ultrasound data from the two or more ultrasound transducer arrays, the ultrasound data comprising information related to one or more of: image quality, image quality metrics, image accuracy, the spinal anatomy, a position or track of the one or more inserted medical instruments, or motion created by and/or from intraspinal blood flow.

13) The system of claim 1, further comprising an integrated medical instrument guide located between at least two of the two or more ultrasound transducer arrays, the integrated medical instrument guide defining a physical gap providing a midline or paramedian trajectory for insertion, guidance, tracking, or combinations thereof, of the one or more medical instruments in-plane with the overlapping acoustic imaging region.

14) The system of claim 13, wherein the one or more medical instruments are one or more of a needle, a catheter, a trocar, an ablation instrument, a cutting instrument, or a therapy applicator.

15) The system of claim 1, wherein the configured processor is further operative to adaptively reconfigure a direction of sound propagation from at least one of the two or more ultrasound transducer arrays to optimize or enhance system sensitivity to the one or more medical instruments based on output of a medical instrument detection sensor and/or algorithm.

16) The system of claim 1, wherein the probe housing is configured to accept the one or more medical instruments in a lateral separation space between two or more of the two or more ultrasound transducer arrays.

17) The system of claim 16, wherein the one or more medical instruments is one or more focused ultrasound therapy transducers.

18) The system of claim 1, wherein the two or more ultrasound transducer arrays do not make direct physical contact with the patient contact surface.

19) The system of claim 1, wherein the configured processor is further operative to control a propagation direction of sound waves from the two or more ultrasound transducer arrays at one or more non-zero angles relative to (a) a central axis of the probe housing, (b) one or more ultrasound transducer arrays of the two or more ultrasound transducer arrays, (c) the two or more ultrasound transducer arrays, or (d) combinations thereof, using automated electronic beam steering.

20) The system of claim 1, wherein the configured processor is further operative to direct or re-direct transmitted energy along an axis that minimizes sound travel distance to an epidural space relative to (a) the probe housing, (b) one or more ultrasound transducer arrays of the two or more ultrasound transducer arrays, (c) the two or more ultrasound transducer arrays, or (d) combinations thereof.

21) The system of claim 1, wherein the configured processor is further operative to adaptively reconfigure a direction of sound propagation of at least one of the two or more ultrasound transducer arrays to optimize or enhance system sensitivity to an intraspinal blood flow based on output of a blood flow sensor and/or detection algorithm.

22) The system of claim 1, wherein the configured processor is further operative to adaptively reconfigure a direction of sound propagation of at least one of the two or more ultrasound transducer arrays to optimize or enhance system sensitivity to a posterior complex based on output of an anatomical detection sensor and/or algorithm.

23) The system of claim 1, wherein the configured processor is further operative to adaptively reconfigure a direction of sound propagation of at least one of the two or more ultrasound transducer arrays to optimize or enhance system sensitivity to an anterior complex based on output of an anatomical detection sensor and/or algorithm.

24) The system of claim 1, wherein the configured processor is further operative to adaptively reconfigure a direction of sound propagation of at least one of the two or more ultrasound transducer arrays to modify one or more angles of acoustic incidence from the at least one of the two or more ultrasound transducer arrays, relative to one or more of the spinal anatomy, a spinal anatomical target, or the one or more medical instruments.

25) The system of claim 1, wherein the configured processor is further operative to adaptively reconfigure a direction of sound propagation of at least one of the two or more ultrasound transducer arrays to modify a field of view or to adjust an extent, an amount, or a percentage, of one or more overlapping regions between imaging planes of the two or more ultrasound transducer arrays.

26) The system of claim 1, wherein the configured processor is further operative to selectively combine ultrasound image data from the two or more ultrasound transducer arrays using weighted ultrasound image fusion to produce a compound image or video.

27) The system of claim 26, wherein the compound image or video are obtained using multiple configurations of sound wave propagation direction from at least one of the two or more ultrasound transducer arrays.

28) The system of claim 1, wherein each ultrasound transducer array of the two or more ultrasound transducer arrays is rotated within the probe housing at angles between about 10° and about 45° relative to the patient contact surface contacted by the probe housing.

29) The system of claim 1, wherein the probe housing further comprises internal acoustic absorption materials positioned to absorb acoustic energy reflected within the probe housing.

30) The system of claim 1, wherein the configured processor is further operative to adaptively generate, move, or change a location of the one or more virtual apex point locations, adaptively select or adjust one or more acoustic transmission powers, adaptively select or adjust one or more acoustic transmission angles, adaptively select or adjust the one or more transmission axes, adaptively select or adjust beam steering angles, adaptively select or adjust virtual apex geometry, or combinations thereof, based on an analysis of ultrasound data, image quality feedback derived from the ultrasound data, image quality metrics derived from the ultrasound data, or combinations thereof.

31) The system of claim 30, wherein the configured processor is further operative to utilize a pre-acquired patient anatomical model to optimize or enhance the adaptive generation, movement, or changing of the location of the one or more virtual apex point locations, the adaptive selection or adjustment of the one or more acoustic transmission powers, the adaptive selection or adjustment of the one or more acoustic transmission angles, the adaptive selection or adjustment of the one or more transmission axes, the adaptive selection or adjustment of the beam steering angles, the adaptive selection or adjustment of the virtual apex geometry, or combinations thereof.

32) The system of claim 30, wherein the configured processor is further operative to utilize an artificial intelligence or machine learning model that receives as input the ultrasound data, for producing an output that optimizes or enhances the adaptive generation, movement, or changing of the location of the one or more virtual apex point locations, the adaptive selection or adjustment of the one or more acoustic transmission powers, the adaptive selection or adjustment of the one or more acoustic transmission angles, the adaptive selection or adjustment of the one or more transmission axes, the adaptive selection or adjustment of the beam steering angles, the adaptive selection or adjustment of the virtual apex geometry, or combinations thereof.

33) The system of claim 1, wherein at least one of the two or more ultrasound transducer arrays are selectively operated in Doppler mode, elastography mode, or both, so that the configured processor is further operative to dynamically evaluate tissue properties of the spinal anatomy along a medical instrument insertion path.

34) The system of claim 1, wherein the configured processor is further operative to provide virtual medical instrument guidance images or information during neuraxial anesthesia procedures, including epidural injections, spinal anesthesia, or lumbar punctures.

35) A computer-implemented method of ultrasound imaging for visualizing spinal anatomy, the method comprising:
positioning an ultrasound imaging probe housing comprising two or more ultrasound transducer arrays on, near, or against a patient contact surface, wherein each ultrasound transducer array of the two or more ultrasound transducer arrays is oriented at a rotation angle within the ultrasound imaging probe housing such that central acoustic axes of the two or more ultrasound transducer arrays intersect to define an overlapping acoustic imaging region of the spinal anatomy;
transmitting acoustic signals from each ultrasound transducer array into the spinal anatomy by using a processor operative to automatically steer acoustic beams from each ultrasound transducer array, the automatic steering determined by the processor generating, and optionally adjusting, a virtual apex point location, wherein acoustic beam transmission axes are automatically selected by the processor, and wherein the automatically selected acoustic beam transmission axes are outside of or out of alignment with each ultrasound transducer array's central acoustic axis, as defined by each ultrasound transducer array's geometry;
receiving ultrasound data; and
acquiring and displaying ultrasound image or video of all or a part of the spinal anatomy, one or more inserted medical instruments, or both.

36) The method of claim 35, wherein, based on the received ultrasound data, the method further comprises: (a) automatically adjusting the automatic steering of the acoustic beams from each ultrasound transducer array, (b) automatically adjusting the virtual apex point location, (c) automatically adjusting the selected acoustic beam transmission axes, or (d) combinations thereof, to produce a visualization, either virtual or real, of the spinal anatomy.

37) The method of claim 35, wherein the displayed image or video comprises an overlaying of the received ultrasound data from the spinal anatomy, the one or more inserted medical instruments, or both, with a virtual representation of the spinal anatomy, a virtual representation of the one or more medical instruments, or both the virtual representation of the spinal anatomy and the virtual representation of the one or more medical instruments, or an overlaying of the virtual representation of the spinal anatomy, the virtual representation of the one or more medical instruments, or both the virtual representation of the spinal anatomy and the virtual representation of the one or more medical instruments, with the received ultrasound data from the spinal anatomy, the one or more inserted medical instruments, or both.

38) The method of claim 35, further comprising generating and displaying a compounded ultrasound image or video by automatically selectively fusing all or parts of the received ultrasound data, thereby enhancing visualization of the spinal anatomy, the one or more inserted medical instruments, or both the spinal anatomy and the one or more inserted medical instruments.

39) The method of claim 35, wherein the two or more ultrasound transducer arrays are physically separated by at least about 1 mm.

40) The method of claim 35, further comprising at least one acoustically transmissive standoff layer located between at least one ultrasound transducer array of the two or more ultrasound transducer arrays and the patient contact surface.

41) The method of claim 40, wherein the acoustically transmissive standoff layer matches or substantially matches an acoustic impedance within ±50% of soft tissue, and wherein the acoustically transmissive standoff layer includes an angled patient interface configured to reduce internal acoustic reverberations.

42) The method of claim 35, wherein at least one of the virtual apex point location or the selected acoustic beam transmission axes are adjusted based on real-time or substantially real-time analysis of the received ultrasound data to optimize or enhance visibility of the spinal anatomy, including anatomical targets, the one or more inserted medical instruments, or both the spinal anatomy and the one or more inserted medical instruments.

43) The method of claim 35, further comprising identifying intraspinal blood flow or tissue properties by processing the received ultrasound data, wherein the received ultrasound data comprises one or more of Doppler imaging information or elastography imaging information.

44) The method of claim 35, further comprising adaptively modifying the acoustic beam transmission axes based on real-time or substantially real-time anatomical feature detection provided by an anatomical feature detection sensor, a machine learning algorithm, or both.

45) The method of claim 35, further comprising comparing and/or contrasting one or more acquired ultrasound images to a pre-acquired anatomical model to optimize or enhance (a) the automatic adjustment of the automatic steering of the acoustic beams from each ultrasound transducer array, (b) the automatic adjustment of the virtual apex point location, (c) the automatic adjustment of the selected acoustic beam transmission axes, or (d) combinations thereof.

46) The method of claim 35, wherein the computer-implemented method of ultrasound imaging for visualizing spinal anatomy is used in interventional procedures selected from a group consisting of: lumbar punctures, epidural injections, nerve stimulation, ablation therapies, and chronic pain therapy injections.

47) A non-transitory computer-readable medium storing executable program instructions which, when executed by at least one processor, cause the at least one processor to perform a method of ultrasound imaging for visualizing spinal anatomy, the method comprising:
positioning the ultrasound imaging probe housing comprising two or more distance-separated ultrasound transducer arrays on, near, or against a patient contact surface, wherein at least one first ultrasound transducer array of the two or more ultrasound transducer arrays is oriented at a rotation angle within the probe housing such that a central acoustic axis of the at least one first ultrasound transducer array of the two or more ultrasound transducer arrays intersects with a central acoustic axis of a second at least one ultrasound transducer array of the two or more ultrasound transducer arrays, to define an overlapping acoustic imaging region;
automatically controlling acoustic signals transmitted from the two or more ultrasound transducer arrays into the spinal anatomy by automatically steering acoustic beams from the first at least one ultrasound transducer array and the second at least one ultrasound transducer array, using a virtual apex transmit aperture technique, the technique comprising automatically selecting acoustic beam transmission axes that are outside of the first at least one ultrasound transducer array's central axis and the second at least one ultrasound transducer array's central axis, as defined by the first at least one ultrasound transducer array's geometry and the second at least one ultrasound transducer array's geometry, thereby generating a virtual apex point using the first and the second distance-separated ultrasound transducer arrays; and
receiving and displaying ultrasound data related to the spinal anatomy, one or more inserted medical instruments inserted into the spinal anatomy, or both the spinal anatomy and the one or more inserted medical instruments.

48) The non-transitory computer-readable medium of claim 47, further comprising sending control signals to and from the two or more distance-separated ultrasound transducer arrays, wherein the first at least one ultrasound transducer array and/or the second at least one ultrasound transducer array comprise at least one acoustically transmissive standoff layer positioned between the first at least one ultrasound transducer array and the patient contact surface and/or between the second at least one ultrasound transducer array and the patient contact surface.

49) The non-transitory computer-readable medium of claim 48, wherein the at least one acoustically transmissive standoff layer matches or substantially matches an acoustic impedance within ±50% of soft tissue, and wherein the first at least one ultrasound transducer array and/or the second at least one ultrasound transducer array include an angled patient interface configured to minimize acoustic reverberations in the probe housing, in one or more of the two or more ultrasound transducer arrays, or combinations thereof.

50) The non-transitory computer-readable medium of claim 47, wherein the first at least one ultrasound transducer array and/or the second at least one ultrasound transducer array are distance-separated by at least about 1 mm.

51) The non-transitory computer-readable medium of claim 47, further comprising generating and displaying compounded ultrasound images or videos by selectively fusing ultrasound information acquired directly or indirectly from the two or more ultrasound transducer arrays, thereby enhancing visualization of the spinal anatomy and/or the one or more inserted medical instruments.

52) The non-transitory computer-readable medium of claim 47, further comprising adaptively adjusting at least one of beam steering angles or virtual apex geometry based on real-time or substantially real-time analysis of the received ultrasound data to optimize or enhance the visualization of the spinal anatomy, visualization of anatomical targets, visualization of the one or more inserted medical instruments, or combinations thereof.

53) The non-transitory computer-readable medium of claim 47, wherein the instructions further cause the processor to identify intraspinal blood flow or tissue properties by processing the received ultrasound data, and wherein the data includes Doppler imaging information and/or elastography imaging information.

54) The non-transitory computer-readable medium of claim 47, wherein the instructions further cause the processor to adaptively modify beam steering angles, the acoustic beam transmission axes, or both, based on real-time or substantially real-time spinal anatomy detection provided by a spinal anatomy detecting sensor and/or machine learning algorithm.

55) The non-transitory computer-readable medium of claim 47, wherein the instructions further cause the processor to compare and/or contrast the received ultrasound data, the overlapping acoustic imaging region, ultrasound images, or combinations thereof, to a pre-acquired anatomical model for optimizing or enhancing beam steering angles, virtual apex geometry, the acoustic beam transmission axes, the virtual apex point, a virtual apex point location, or combinations thereof.

56) The non-transitory computer-readable medium of claim 47, wherein the ultrasound imaging for visualizing the spinal anatomy is used for an interventional procedure selected from the group consisting of lumbar punctures, epidural injections, nerve stimulation, ablation therapies, and chronic pain therapy injections.
